(19) United States
(12) Patent Application Publication
(10) Pub. No.: US 2015/0291706 A1
(43) Pub. Date: Oct. 15, 2015

(54) Photoreactive Derivative of Hyaluronic Acid, Method of Preparation Thereof, 3D.Crosslinked Derivative of Hyaluronic Acid, Method of Preparation and Use Thereof

(71) Applicant: Contipro Biotech s.r.o., Dolní Dobrouc (CZ)

(72) Inventors: Tomas Bobula, Svit (SK); Robert Pospisil, Spojil (CZ); Radovan Buffa, Humenne (SK); Jana Ruzickova, Zamberk (CZ); Martina Moravcova, Letohrad (CZ); Pavel Klein, Dolni Dobrouc (CZ); Vladimir Velebny, Zamberk (CZ)

(21) Appl. No.: 14/647,185

(22) PCT Filed: Nov. 26, 2013
(86) PCT No.: PCT/CZ2013/000155

§ 371 Date: May 26, 2015

(30) Foreign Application Priority Data
Nov. 27, 2012 (CZ) PV 2012-844

(51) Int. Cl. C08B 37/08; A61Q 17/04; A61K 8/73; A61Q 19/00; A61L 27/20; A61L 26/00

(52) U.S. Cl. CPC C08B 37/0072; A61L 27/20; A61L 26/0023; A61K 8/735; A61Q 19/00; A61Q 17/04; A61L 2300/236

(57) ABSTRACT

The subject-matter of the invention is a photoreactive derivative of hyaluronic acid (formula I) and the method of preparation thereof, where first an aldehyde derivative of hyaluronic acid is prepared, oxidized in the position 6 of the glucosamine cycle and then the oxidized derivative reacts with an amine carrying a photoreactive species, for example 1-(2-aminoethyl)pyridine-2(1H)-one, in the presence of a reducing agent, forming a photoreactive derivative. The prepared photoreactive derivative may be then photocrosslinked, wherein the reaction is based on [4+4] photocycloaddition. Moreover, the invention relates to a 3D-crosslinked derivative of hyaluronic acid (formula II) which exhibits an increased hydrolytic stability and improved sorption properties, with the possibility of a further design of the physical properties thereof according to the requirements of the final applications, and moreover, to the use thereof in tissue engineering, regenerative medicine, medical agents or formulations or cosmetics.

### FIELD OF THE ART

The invention relates to the preparation of the 3-D structure of hyaluronic acid prepared by the photochemical crosslinking. The methodology is based on the intermolecular photocycloaddition or photodimerization of a suitable chromophore incorporated into a polymer chain of hyaluronic acid. The photoreactions are carried out in the absence of an inert atmosphere, the reactions proceed in air, at room temperature, without the necessity of using an organic solvent, without any isolation process needed for the desired product, or any disposal of the side by-products. The product of the photochemical reaction is a dimer structure (the so-called crosslink) of the low-molecular chromophore bound to the hyaluronic acid polymeric chain. By these means, the formation of 3-D crosslinked structure of hyaluronic acid is ensured, exhibiting a substantially lower solubility and higher stability in an aqueous medium than the initial material.

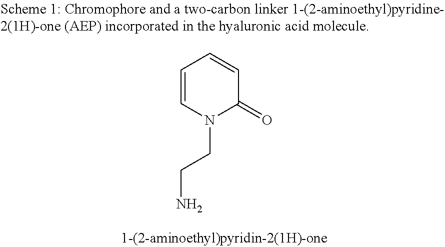

### PRIOR ART

Hyaluronic acid is a natural heteropolysaccharide of the glycosamino glycans, composed of D-glucuronic and N-acetyl-D-glucosamine subunits which are bound to each other by β(1→3) and β(1→4) O-glycosidic bonds. Hyaluronic acid occurs naturally in a number of connective tissues, synovial liquid, skin and in the cartilage (Smeds K. A., Grinstaff M. W. 2001. J Biomed Mater Res 54: 115). Hyaluronic acid is prone to an enzymatic degradation (Burdick J. A., Chung C., Jia X., Randolph M. A. and Langer R. 2005. Biomacromolecules 6: 386) and plays an important role in hydration of tissues, cell differenciation (Park Y. D., Tirelli N., Hubbell J. A. 2003. Biomaterials 24: 893), in the wound healing (Leach J. B. and Schmidt C. E. 2003. Biotechnol Bioeng. 82: 578), angiogenesis (Leach J. B. and Schmidt C. E. 2005. Biomaterials 26: 125) and in the treatment of chronic diseases (Jia X. Q., Burdick J. A., Kobler J., Clifton R. J., Rosowski J. J., Zeitels S. M., Langer R. 2004. Macromolecules 37: 3239).

Hyaluronic acid is interesting from the biomaterial applications point of view especially in tissue engineering. The functional groups (OH, COOH) in the polymeric structure enable a subsequent chemical derivatization (e.g. selective oxidation Buffa R., Kettou S. and Velebný V., PV 2009-835, 2009-836) leading to a chemical (Burdick J. A. and Prestwich D. G. 2011. Adv Mater 23, H41) or photochemical crosslinking, giving rise to the hydrolytically-stable covalent bonds (Seidlits S. K., Khaing Z. Z., Petersen R. R., Nickels J. D., Vanscoy J. E., Shear J. B., Christine E. Schmidt Ch. E. 2010. Biomaterials 31: 3930).

Photocycloaddition Reactions of HA

Ones of the most frequently used photochemical reactions leading to the crosslinking of a macromeric HA chain are the so-called [2+2] photocycloadditions or [2+2] photodimerizations. During these two intermolecular reactions, transformation of two unsaturated π-bonds to saturated σ-bonds occurs, that results in the formation of 4-membered cyclobutane ring (crosslink) with its side chains bound to the biopolymeric structure (scheme 2).

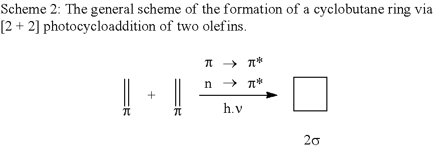

In case of polysaccharides, there exist many chromophores containing a conjugated double bond, which undergo [2+2] photocycloaddition upon excitation by the UV light. These photoreactive compounds include: acrylic acid, methacrylic acid, furylacrylic acid, thienylacrylic, fumaric acid, maleic acid, sorbic acid, cinnamic acid including the p-amino derivative thereof, maleinimide and alkyl and aryl derivatives thereof, pyrimidine bases (uracil, thymin and cytosin), pyran-2-one, coumarin, psoralen, trans-chalcons, trans-stilbens and metoxyl derivatives thereof and quarternary pyridinium salts (trans-4-stryrylpyridinium halides).

Applications of [2+2] Photocycloaddition Reactions

A complex patent of Seikagaku Corporation, JP, (Matsuda T., Moghaddam M. J, Sakurai K. 1993, EP0554898B1) was published in 1993. The authors described the preparation of the photoreactive heteropolysaccharides, especially GAG (from the English glucosaminoglycans), including hyaluronic acid. They intended to use the photochemically crosslinked hyaluronic acid based on cinnamic acid in the cardiomorphogenesis.

In the patent (Motani Y., Seikagaku Corporation, JP, 1997, EP0763754A2) the authors presented the derivatives of hyaluronic acid substituted by trans-cinnamic acid. The 3-D crosslinked products were used in contact lenses. The crosslinked derivatives were the transparent and compact hydrogels applicable on the surface of an eyeball. The authors claimed the shape stability, antiadhesive properties, well-defined mechanical and absorption properties (20-99% of the gel volume was water-based) of the used materials.

The patent document (Waki M. and Motani Y., Seikagaku Corporation, JP, 2000, US006025444) developed and optimized the use of trans-cinnamic acid. The authors succeeded in an explanation for the reason of the low reactivity thereof in the structure of hyaluronic acid. They gave the reason to the competitive photochemical reaction—photoisomerization. According to the authors, the concentration of a selected photoreactive derivative of hyaluronic acid had a crucial influence on the ratio between an occurring photocycloadduct and the competitor thereof in the form of a photochemically inactive cis-isomer of cinnamic acid.

The complex patent application (Sato T., 2003, Seikagaku Corporation, JP, EP1607405B1) claimed two photoreactive groups, which are trans-cinnamic acid and a pyrimidine base—thymine. The authors defended the inventive step by the irradiation of the frozen photoreactive derivatives of the biopolymers, or by the addition of a chelating agent, detergent into an irradiated solution, which leads to the formation of scaffolds suitable for the proliferation of stem cells.

In the year of 2006, the patent (Miyamoto K., Kurahashi Y., Seikagaku Corporation, JP, 2006, EP1217008B1) was granted in the field of photochemistry of trans-cinnamic acid attached to hyaluronic acid. The authors saw the inventive step of their experiments in the application of an alkaline conditions during the photochemical reaction. The modified pH (7.2 to 11.0), ideally (7.5-10.0) of the reaction had an essential influence on the solubility (hydrophilicity) of hyaluronic acid and also on the character of the secondary and tertiary structure thereof. This resulted in much more efficient self-assembly of the photoreactive groups where the higher quantum yields were subsequently achieved.

The patent document (Miyamoto K., Yasuda Y., Seikagaku Corporation, JP, 2008. EP1905456A1, international application, 2007, WO2007/004675) presented the photoreactive derivatives of HA derived from trans-cinnamic acid, containing a covalent incorporated medicinal substance (preferably an antiphlogistic agent). The sol-gel transition of the hyaluronic acid derivatives and the parameters of the obtained hydrogel reflected subcutaneous application (needle 20 to 25) with the pressure (0.5-5 kg/cm²) into the organism with time-designed release of the medicinal substance at the location of an application. The medicinal substances were especially non-stereoidal inflammatory drugs (NAID) such as naproxen, ibuprofen, flubiprofen, felbinac, etodolac or actarit.

The international patent application (Francotte E., CIBA-Geigy, CH, 1996. WO96/27615 patent family: 2000, U.S. Pat. No. 6,011,149, 2002, EP08137546B1) provides an interesting and a useful application of [2+2] photocycloaddition reactions in the field of a design of the new stationary phases for column chromatography to efficiently separate an anomeric mixture. The author introduced the dimerisation reaction of a substituted maleinimide attached by a carbamate bond to a polysaccharide chain bearing the required chiral information. The patent claimed several types of polysaccharides such as cellulose, amylose, chitosan, dextran, xylan or inulin.

A comprehensive publication from 1989 (Katritzky A. R., Dennis N., 1989. Chem Rev 89: 827) discussed in detail the (photo)chemistry of the cycloaddition reactions of 6-membered heterocyclic compounds. The authors described, by means of cited references to original literature, the scope of [2+2] photocycloaddition reactions of nitrogen bases and other chromophores derived from chinolin-1-oxide, pyran-2-one, coumarin, substituted chromone, dihydropyridine and dihydropyran-2,4-dione.

Much effort was devoted to the applications of pyrimidine bases (cytosine, thymine, uracil) in the field of the photodimerization reactions, which resulted in a number of patents such as (Grasshoff J. M, Taylor D. L., Warner N., Polaroid corporation, UK, 1995. U.S. Pat. No. 5,455,349); (Matsuda T., Nakao H., Seikagaku Kogyo, JP, 2000. U.S. Pat. No. 6,075,066); (Sato T., Seikagaku Corporation, JP, 2003. EP1369441A1); (Warner J. C., Morelli A., Ku M. Ch., University of Massachusetts, 2005. US20050266546A1); (Warner J. C., Cannon A. S., Raudys J., Undurti A., University of Massachusetts, 2009. U.S. Pat. No. 7,550,136). Their applications were directed to the field of a cosmetic industry, optics, tissue engineering and regenerative medicine.

[2+2] Photocycloadditions give rise to a saturated cyclobutane ring (4-membered without any double bond) as a product in which no further chemical modification is possible and the structure does not represent any biological motive. Contrary, the presented invention including the [4+4] photocycloaddition is original and it provides several advantages. The [4+4] photocycloaddition affords an unsaturated β-lactame cycle (8-membered with two double bonds) as a product, which can undergo a further chemical modification. Moreover the cyclooctadiene crosslink is considered to be an interesting biological motive introduced into the structure of the crosslink (Holten K. B., Onosuko E. M. 2000., American Family Physician 62: 611; Elander R. P., 2003. Applied Microbiology and Biotechnology 61: 385).

Another advantage of [4+4] photocycloaddition, compared to the other approaches based on the photodimerization strategy, is the unique structure of a formed crosslink. The discussed character, as opposed to the [2+2] photocycloaddition reaction where only a 4-membered and a saturated cyclobutane ring is formed, enables the formation of 8-membered cycle containing two multiple bonds. The isolated double bonds in the crosslink are easily accessible for an additional chemical modification (oxidation, reduction, or addition). The [4+4] photocycloadditions have not been used for the photochemical crosslinking of hyaluronic acid so far. Discussed [4+4] photocycloadditions proceed in a solid phase, and therefore, they do not require any solvent, any degassing of the reaction mixture, any complicated preparation of the sample and they do not depend on the solution parameters, such as the concentration or viscosity. The great advantage of a presented strategy is the reaction without any toxic solvents, high selectivity of the reaction, no need for both an inert atmosphere and the isolation of a final product, which significantly reduces costs and facilitates the experiment itself. Moreover, the effectiveness of the process is substantially increased (isolation, separation, purification, the amount of waste). These factors are much desirable from the industrial point of view.

Besides, an important innovation step according to the invention is also the character of the photoreactive group based on 2-pyridone. Many chromophores exhibit an increased sensibility towards oxygen and they easily undergo an undesirable ozonolysis, or very reactive radicals are formed which cause the photodegradation of the biopolymer. Therefore in such cases, the photochemical reactions cannot be carried out freely opened to the air atmosphere. First of all, the degassing (deoxygenation) of the reaction mixture must take place, followed by the flow of an inert atmosphere must be ensured and only after that is it possible to proceed with the photochemical reaction itself. Our photoreactive group does not require this advance preparation because it is not sensitive to oxygen (Sieburth S. M, Cunard T. N., 1996. Tetrahedron 52: 6251; Dilling W. L., Mitchell A. B., 1973. Mol. Photochem. 5:, 371; Matsushima R., Terada K. 1985. J. Chem. Soc. Perkin Trans. 2, 1445). Its stability reflects the conjugation thereof which substantially decreases the susceptibility of the double bonds to the degradation thereof. That means that the solution according to the invention will be substantially simplified and will be more advantageous economically, compared to the state of the art in the field of the photocrosslinking of polysaccharides.

### Subject-Matter of the Invention

The subject-matter of the invention is a method of photocrosslinking of the photoreactive derivatives of hyaluronic acid based on [4+4] photocycloadditions. These reactions enable the formation of a transversal bond (crosslink) and thereby form the crosslinked structures of hyaluronic acid. Another advantage of [4+4] photocycloadditions, compared to the other solutions based on the photodimerization strategy, is the character of the structure of the formed crosslink. Said character, as opposed to the [2+2] photocycloaddition reaction where only a 4-membered and saturated cyclobutane ring is formed, enables the formation of 8-membered ring containing two multiple bonds. The isolated double bonds in such configuration are easily accessible to an additional chemical modification (oxidation, reduction, or addition).

Moreover, the use of 2-pyridone as a photoreactive group is not so sensitive to the atmospheric oxygen, which greatly simplifies the experimental realization compared to those with other chromophores. The reason is a partial delocalization of π-electrons of the conjugated multiple bonds which results from the resonance of this heterocycle. Of course, the invention is not limited just to 2-pyridone and its derivatives. Potentially useful chromophores include e.g. acridizinium salts, anthracene, 2-pyrones, benzofurans and the like.

The photocrosslinked derivative of hyaluronic acid is characterized by the modification of its physical properties, represented by an increased hydrolytic stability and a limited solubility in an aqueous media. Further, it is characterized by that in an aqueous medium it swells, forms hydrogels, insoluble particles, exhibits sorption properties and ensures retention of liquids, dyes, optionally biologically active substances.

The presented approach of the formation of 3-D crosslinked products of hyaluronic acid is composed of three steps (scheme 1). The preparation of the photoreactive derivative of hyaluronic acid starts from the oxidized form thereof (step 1, scheme 1) and an amine carrying the target chromophore. A hydrolytically instable imine is formed in the reaction mixture, which is directly reduced in situ by a hydride to a hydrolytically stable secondary amine (step 2, scheme 1). For this purpose, N-alkylated derivative of 2-pyridone (1-(2-aminoethyl)pyridine-2(1H)-one) (hereinafter just AEP) was synthesized by a selective N-alkylation of pyridine-2(1H)-one with 2-(Boc-amino)ethylbromide. The last step is the photocrosslink itself (step 3, scheme 1) of the prepared HA derivatives, leading to the formation of 3-D crosslinked products. The photocrosslink is initiated by the UVB light, takes place in a solid phase, i.e. without any solvent, chemical catalysis or inert atmosphere. This kind of photoreaction is classified as [4+4] photocycloaddition or [4+4] photodimerization

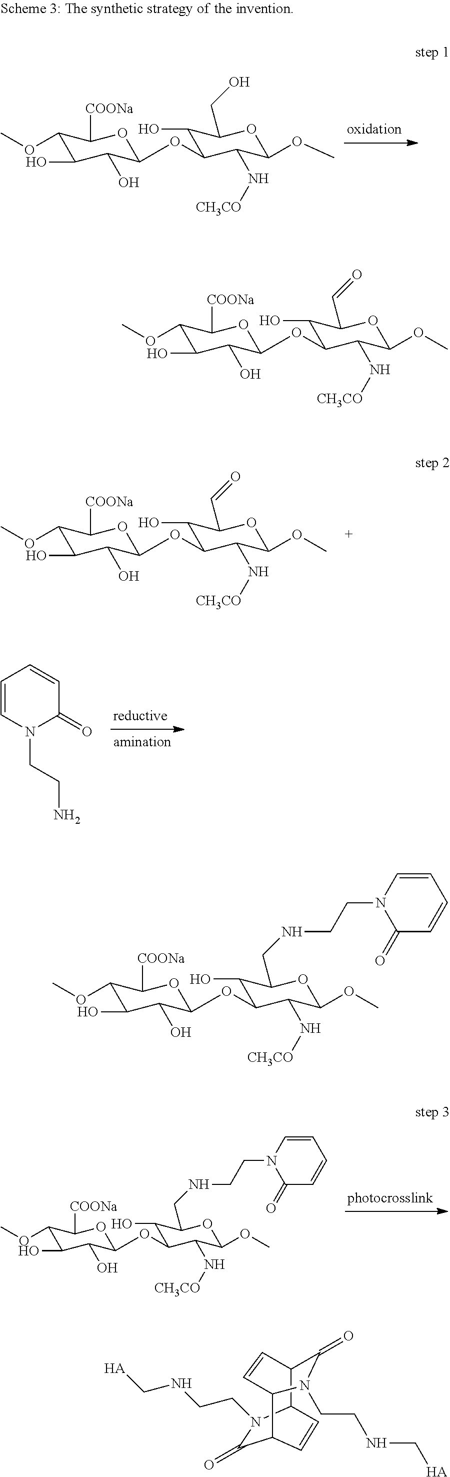

In particular, the invention relates to the photoreactive derivative of hyaluronic acid according to the formula (I), wherein R represents hydrogen or an alkali metal cation:

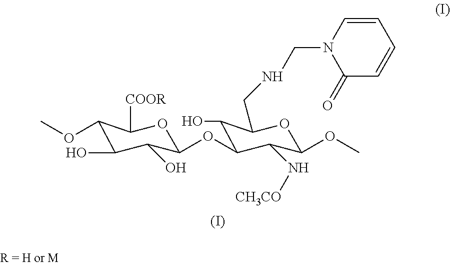

Hyaluronic acid or an inorganic salt thereof has the molecular weight within the range of 1.10⁴ to 5.10⁶ g.mol⁻¹.

Further, the invention relates to the method of preparation of the derivative according to the formula (I), wherein first an aldehyde of hyaluronic acid formed in the position 6 of the glucosamine cycle is prepared and then the oxidized derivative is reacted with an amine carrying the photoreactive species in the presence of a reductive agent, forming the photoreactive derivative. The preparation of the aldehydic derivative of hyaluronic acid selectively oxidized in the position 6 of the glucosamine cycle may be performed by the oxidation agent Dess-Martin periodinane in an aprotic medium or by a TEMPO radical with NaClO in an aqueous medium. Subsequently, the aldehyde of hyaluronic acid reacts with the amino group of the amine carrying the photoreactive species (i.e. with the chromophore with the bound two-carbon based linker) forming an imine which is directly reduced in one step, in the presence of a reducing agent NaBH₃CN in an aqueous medium or in the water-organic solvent system, to a secondary amine. The amine bearing the photoreactive group may be e.g. 1-(2-aminoethyl)pyridine-2(1H)-one.

In another aspect, the invention relates to the method of preparation of 3D crosslinked derivatives of hyaluronic acid wherein the photoreactive derivative according to the formula (I) is treated by electromagnetic radiation within the wavelengths of 280-315 nm. The photoreactive derivative may be in a form of a powder, a lyophilizate, a thin film, a nanofibrous or microfibrous structure.

Moreover, the invention relates to the 3D crosslinked derivative of hyaluronic acid according to the formula (II):

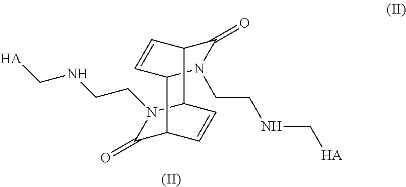

as well as to the use thereof for tissue engineering, regenerative medicine, medical agents or formulations or cosmetics.

Therefore, the prepared 3D crosslinked structures of hyaluronic acid exhibit an increased hydrolytic stability, good sorption properties and provide a space for further design of physical properties thereof depending on the actual interdisciplinary needs. This implies individual applications such as: for tissue engineering (scaffolds, fillers, drug delivery systems), for regenerative medicine (supportive nano- or micro-structures for the growth of the cells stem cells or differentiated cells such as: chondrocytes, fibroblasts, neurocytes and the like), wound healing applications (nano- or micro-structures, woven fabrics, knitted fabrics may be used for the production of biodegradable bandages for surface wounds with controlled release of biologically active substances) and also wide applications in cosmetics (such as for the production of facial masks, additive to sun lotions with a preventive or regenerative effect).

### BRIEF DESCRIPTION OF DRAWINGS

FIG. 1 represents the comparison of three different forms of one type of the photoreactive derivative of hyaluronic acid (Mw=25 kDa, DS=18%) before UV irradiation. Micrographs of SEM analysis of the photocrosslinked derivatives (t=1 h, E=23400 mJ.cm⁻²) after 48-hour swelling in PBS (pH=7.4) at 20° C. Atop—thin film (T): range 2 mm, 500 μm, 2 μm. Center—lyophilizate (L): range (500, 50, 10) μm. Alow—nanofibrous layer (N): range: (500, 50, 10) μm.

FIG. 2 represents a micrograph of SEM analysis of the photoreactive derivative of hyaluronic acid (25 kDa, DS=18%) in the form of a nanofibrous layer, range 10 μm, magnification 3.22 kx (k=1000), the fibre diameters 189±50 nm.

FIG. 3 represents micrographs of SEM analysis of lyophilized, photocrosslinked derivatives of hyaluronic acid (25 kDa, DS=18%, tₑₓₚ=1 h, E=23400 mJ.cm⁻²) in the form of a swelled nanofibrous layer in water for (1 h), scale 20 μm, magnification 2.02 kx, (left). A detailed view, scale 5 μm, magnification 5.54 kx, fibre diameters 314±202 nm (right).

FIG. 4 represents the results of the cell viability test of 3T3 fibroblasts in the environment of the photoreactive derivative of hyaluronic acid (Mw=34 kDa, DS=20%). The growth curve in the percentual representation with respect to the control in time T=0 h (100%). The evaluation by means of MTT method in five repetitions n=6.

FIG. 5 represents test results of the influence of UVA (315-380 nm) on the cell viability of 3T3 fibroblasts. Positive (anthracene) and negative (SDS) control. The evaluation by means of MTT method in 3 repetitions n=3. The concentration of the substances: anthracene (1-30 μg/ml), SDS (1-15 μg/ml), control without additives (100%).

FIG. 6 represents test results of the influence of UVA (315-380 nm) on the cell viability of 3T3 fibroblasts. Evaluation by means of MTT method in five repetitions n=5. The concentration of the photoreactive derivative (Mw=34 kDa, DS=20%)=1, 3, 30, 100, 500, 1000, 5000 μg/ml, control without the derivative (100%).

FIG. 7 represents enzymatic degradation of the photocrosslinked derivatives of hyaluronic acid with respect to (Mw=34 kDa, DS=20%) 1 mg of the sample and expressed by means of the equivalents of glucose hemiacetal.

### EXAMPLES

DS was determined by means of NMR (nuclear magnetic resonance) and calculated according to the following relation: DS=substitution degree=100%*molar amount of the bound substituent/molar amount of all polysaccharide dimers. The calculation is from the relative ratio of the integral values of signals of two diastereotopic hydrogens in the position 6 of the glucosamine subunit, characteristic for the given modification as opposed to the integral of N-acetyl group.

TEMPO radical is 2,2,6,6-tetramethylpiperidinyloxyl radical.

NMR spectra of the samples were measured on BRUKER AVANCE 500 MHz apparatus in D₂O or CDCl₃. Chemical shifts were calibrated to the internal standard of deuterated sodium salt of 3-trimethylsilylpropanoic acid (TSPA). The data were processed by the software Bruker TOPSPIN 1.2 or software Spinworks 3.1.7.

The term equivalent (eq) used herein relates to a dimer of hyaluronic acid, if not indicated otherwise. Percentages are used as weight percentages, if not indicated otherwise.

The molecular weight of the initial hyaluronan (source: Contipro Biotech s.r.o, Dolni Dobrouc, CZ) was determined by SEC-MALLS.

FT-IR spectra were measured within the range of 4000-400 cm⁻¹ as KBr tablets or in the form of a thin film on Nicolet 6700 FTIR spectrometer.

UV-VIS spectra were measured on Shimadzu UV-2401PC apparatus within the range of 200-800 nm and processed by UV Probe software, version 2.00.

The surface morphology of the lyophilized samples was examined by a scanning electron microscope Tescan VEGA II LSU. The samples were measured at 20° C. and evaluated by VegaTC 3.5.2.1 software. (10 kV, working distance 3.4 mm, magnification 1000-20 kx).

The photocrosslink was performed by use of UV Crosslinker CL-1000M (302 nm, 6.75 mW/cm²) according to the methods A-C.

### Example 1

### Oxidation of Hyaluronic Acid with DMP

2% solution of the acid form of hyaluronic acid (2.0 g, 5.29 mmol, Mw=270 kDa) in dry DMSO is prepared. DMP (1.91 g, 4.49 mmol) is added to the resulting solution and the reaction mixture is stirred for 5 hours. Afterwards EtOH (3 ml) is added. The product is ultrafiltrated and lyophilized.

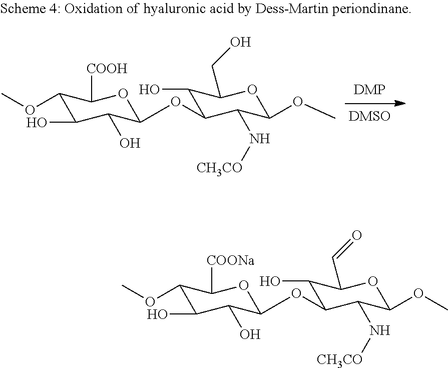

DS=20%, Mw=34 kDa, isolated yield 91%
¹H NMR (D₂O) δ 5.26 (s, 1H, polymer-CH(OH)₂) ppm—geminal diol (hydrated aldehyde)
HSQC (D₂O) crosspeak δ 5.26 ppm (¹H)—90 ppm (¹³C) polymer-CH(OH)₂
FT-IR (KBr) 1740 cm⁻¹—CH═O

### Example 2

### Oxidation of Hyaluronic Acid by Tempo/NaOCl

2% (aq) solution of hyaluronic acid (5.0 g, 12.50 mmol, Mw=950 kDa) is prepared. NaBr (642.5 mg, 6.25 mmol) and Na₂HPO₄.12H₂O (9.71 g, 27.12 mmol) are added. The reaction mixture is stirred for 15 minutes at room temperature. The reaction mixture is cooled to 5° C. Subsequently 4-acetamido-TEMPO (26.7 mg, 0.13 mmol) and NaClO solution (1.47 ml, 6.25 mmol) are added. The reaction mixture is stirred for 2 hours at 5° C. Afterwards EtOH (7.29 ml, 125.0 mmol) is added. The product is ultrafiltrated and lyophilized.

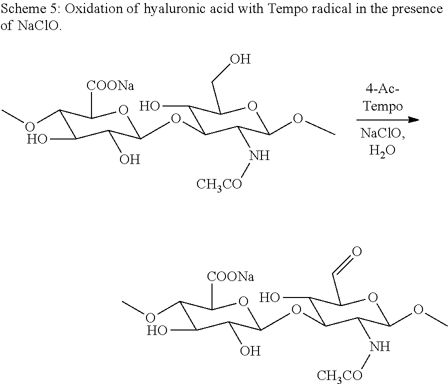

DS=8%, Mw=288 kDa, isolated yield 82%
¹H NMR (D₂O) δ 5.26 (s, 1H, polymer-CH(OH)₂) ppm
HSQC (D₂O) crosspeak δ 5.26 ppm (¹H)—90 ppm (¹³C) polymer-CH(OH)₂
FT-IR (KBr) 1740 cm⁻¹—CH═O

### Example 3

### Synthesis of 1-(2-aminoethyl)pyridine-2(1H)-one (AEP). N-alkylation of pyridine-2(1H)-one with 2-(Boc-amino)ethylbromide

Pyridine-2(1H)-one (100.0 mg, 1.051 mmol) is dissolved in 2 ml of EtOH (dry) in a three-necked flask equipped with a stirrer, cooler and balloon with an inert gas. KOH (66.1 mg, 1.182 mmol) is added to the solution and the reaction mixture is stirred for 30 minutes. Then, 2-(boc-amino)ethylbromide (313.3 mg, 1.398 mmol) is added. The reaction mixture is refluxed for 5 hours. The solvent is evaporated on a vacuum rotary evaporator. The evaporation residue is dissolved in 10 ml of CHCl₃. 25% solution of NH₄OH (10 ml) is added added to the solution. The organic phase is washed with (2×5 ml) H₂O and (1×5 ml) brine (normally used). It is dried over MgSO₄, filtrated, and the solvent is evaporated on a vacuum rotary evaporator. The product is isolated by the column chromatography on Si-gel, by use of the gradient (MeOH, CHCl₃).

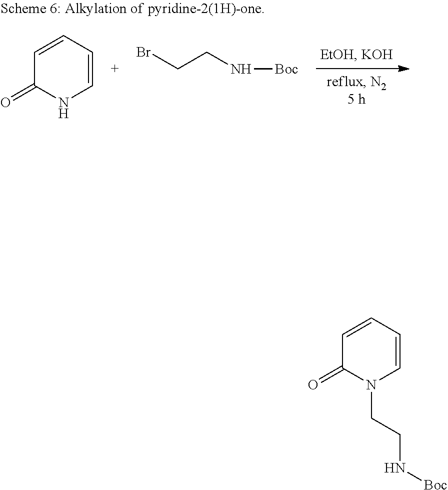

N-alkyl product: tert-butyl 2-(2-oxopyridine-1(2H)-yl)ethylcarbamate, C₁₂H₁₈N₂O₃, Mw=238.283 g/mol, colorless crystals, R_{F} (TB-16-F2)=0.70 (CHCl₃:MeOH/9:1), isolated yield=41%. ¹H NMR (500 MHz, CDCl₃): δ=7.31 (ddd, J=9.0; 6.6; 2.1 Hz, 1H), 7.24-7.26 (m; 1H); 6.54 (d; J=9.0 Hz; 1H); 6.16 (t; J=6.6 Hz; 1H); 5.13 (bs; 1H); 4.07 (t; J=6.0 Hz; 2H); 3.42 (q; J=6.0 Hz; 2H); 1.39 (s; 9H) ppm
¹³C NMR (125 MHz; CDCl₃): δ=162.9; 156.1; 139.8; 138.2; 120.8; 106.2; 79.5; 49.3; 39.8; 28.3 (3C) ppm
O-alkyl product: tert-butyl 2-(pyridine-2-yloxy)ethylcarbamate; C₁₂H₁₈N₂O₃; Mw=238.283 g/mol; colorless viscous oil; R_{F}=0.80 (CHCl₃:MeOH/9:1); isolated yield=5%;
¹H NMR (500 MHz; CDCl₃): δ=8.12 (dd; J=4.9; 1.5 Hz; 1H); 7.55-7.58 (m; 1H); 6.85 (ddd; J=5.9; 5.1; 0.7 Hz; 1H); 6.72 (t; J=8.4 Hz; 1H); 4.95 (bs; 1H); 4.36 (t; J=5.2 Hz; 2H); 3.45 (q; J=5.2 Hz; 2H); 1.44 (s; 9H) ppm
¹³C NMR (125 MHz; CDCl₃): δ=163.5; 155.6; 146.9; 138.7; 116.9; 110.9; 81.1; 65.0; 40.2; 27.8 (3C) ppm

### Example 4

### Deprotection of tert-butyl 2-(2-oxopyridine-1(2H)-yl)ethylcarbamate

Boc-amine (43.0 mg, 0.180 mmol) is dissolved in dichloromethane (300 μl) under inert atmosphere of N₂. TFA (275 μl, 3.6 mmol) is added and the reaction mixture is stirred for 2 hours at room temperature. The excess of trifluoroacetic acid (b.p.=72.4° C.) and dichloromethane is evaporated on a vacuum rotary evaporator and the evaporation residue is neutralised with saturated solution of NaHCO₃. 2 ml of CHCl₃ are added to the aqueous solution. The extract is washed with H₂O (1×2 ml), brine (1×2 ml) and dried over MgSO₄. The reaction mixture is filtrated and is evaporated on a vacuum rotary evaporator.

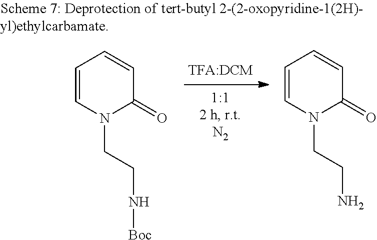 [SMILES: CC(C)(C)OC(=O)NCCn1ccccc1=O ; "TFA:DCM" ; NCCn1ccccc1=O]

1-(2-aminoethyl)pyridine-2(1H)-one, C₇H₁₀N₂O, Mw=138.167 g/mol, yellowish liquid; R_{F}=0.18 (CHCl₃:MeOH/1:1); isolated yield=80%,
¹H NMR (500 MHz; D₂O): δ=7.65-7.68 (m; 2H); 6.66 (d; J=9.5 Hz; 1H); 6.72 (dt; J=6.8; 1.2 Hz; 1H); 4.09 (t; J=6.1 Hz; 2H); 2.99 (t; J=6.1 Hz; 2H) ppm
¹³C NMR (125 MHz; D₂O): δ=167.1; 145.2; 142.2; 122.0; 112.2; 55.1; 42.4 ppm

### Example 5

### Reductive Amination with 2 Equivalents of AEP. The Introduction of a Chromophore into the Biopolymer

The oxidized form of hyaluronan (100.0 mg, 0.265 mmol, DS=20%, Mw=34.4 kDa) is dissolved in 10 ml of distilled water (1% solution). To said solution, AEP (14.6 mg, 0.106 mmol, 2 eq.) is added. The reaction mixture is stirred for 2 hours. Then NaBH₃CN (26.5 mg, 0.425 mmol) is added and the reaction mixture is stirred for additional 12 hours. The final solution is dialysed and lyophilized.

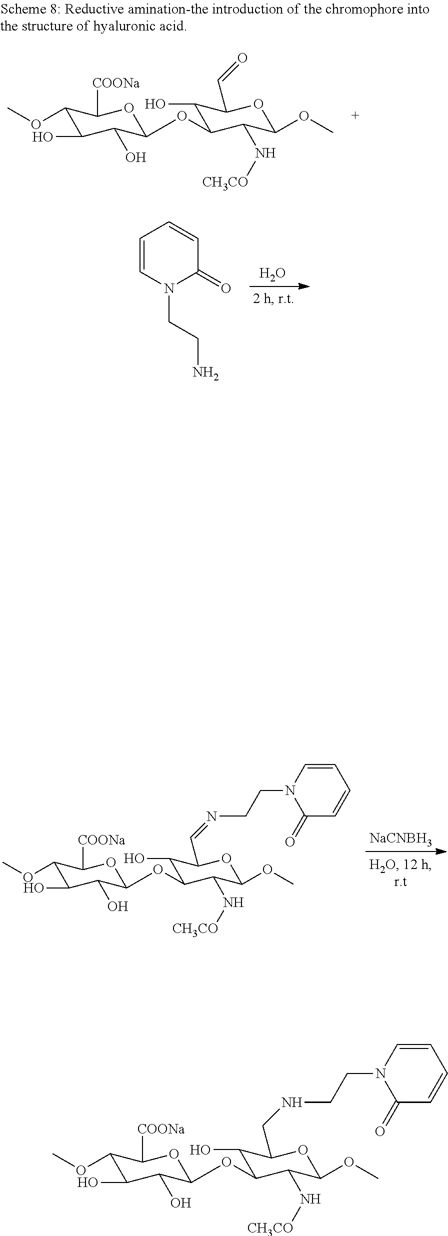

DS=16%; Mw=34 kDa; isolated yield 65%
¹H NMR (D₂O+NaOD) δ 2.78 (bs; 1H; polymer-H6^{a}); 2.99 (bs; 1H; polymer-H6^{b}); 2.94-3.00 (m; 2H; —NHCH₂—); 4.13-4.17 (m; 2H; —NCH₂—); 6.58 (bs; 1H; Hₕₑₜₐᵣ); 6.66 (bs; 1H; Hₕₑₜₐᵣ); 7.64-7.70 (m; 2H; Hₕₑₜₐᵣ) ppm,
H—H COSY (D₂O+NaOD) crosspeak δ 2.78-2.99; 3.00-4.16; 6.58-7.65; 6.66-7.69 ppm
HSQC (D₂O+NaOD) crosspeak δ 2.78 (¹H)-49.0 (¹³C); 2.99 (¹H)-49.0 (¹³C); 3.00 (¹H)-47.4 (¹³C); 4.16 (¹H)-50.0 (¹³C); 6.58 (¹H)-110.2 (¹³C); 6.66 (¹H)-118.1 (¹³C); 7.69 (¹H)-136.4 (¹³C); 7.65 (¹H)-145.0 (¹³C) ppm
DOSY NMR (D₂O+NaOD) log D (2.03 ppm; Me-CO—NH-polymer)˜−10.45 m²/s
   log D (2.78 ppm; polymer-H6^{a})˜−10.45 m²/s
   log D (2.99 ppm; polymer-H6^{b})˜−10.45 m²/s
   log D (3.00 ppm; —NHCH₂—)˜−10.45 m²/s
   log D (4.16 ppm; —NCH₂—)˜−10.45 m²/s
   log D (6.58 ppm; Hₕₑₜₐᵣ)˜−10.45 m²/s
   log D (7.65 ppm; Hₕₑₜₐᵣ)˜−10.45 m²/s
   log D (7.65-7.69 ppm; Hₕₑₜₐᵣ)˜−10.45 m²/s
   log D (4.72 ppm; H₂O)˜−8.6 m²/s
FT-IR (KBr) 1654 cm⁻¹ Nₕₑₜₐᵣ-C═O
UV/vis (0.005%; H₂O) λₘₐₓ=299 nm; Nₕₑₜₐᵣ-C═O

### Example 6

### Reductive Amination with 1 eq of AEP

The oxidized form of hyaluronan (100.0 mg, 0.265 mmol, DS=8%, Mw=288 kDa) is dissolved in 10 ml of distilled water (1% solution). To said solution, AEP (3.1 mg, 0.022 mmol, 1 eq.) is added. The reaction mixture is stirred for 2 hours. Then NaBH₃CN (26.5 mg, 0.425 mmol) is added and the reaction mixture is stirred for further 12 hours. The final solution is dialysed and lyophilized.
DS=3%, Mw=229 kDa, isolated yield 95% (determined by NMR, more details in Example 5)

### Example 7

### Reductive Amination with 2 eq of AEP and 2%_{(aq)} Solution

The oxidized form of hyaluronan (100.0 mg, 0.265 mmol, DS=20%, Mw=34.4 kDa) is dissolved in 5 ml of distilled water (2% solution). To said solution, AEP is added (14.6 mg, 0.106 mmol, 2 eq.). The reaction mixture is stirred for 2 hours. Then NaBH₃CN (26.5 mg, 0.425 mmol) is added and the reaction mixture is stirred for further 12 hours. The final solution is dialysed and lyophilized.
DS=20%, Mw=34 kDa, isolated yield 74% (determined by NMR, more details in Example 5)

### Example 8

### Reductive Amination with 1.5 eq of AEP, Addition of 1 eq of NaHCO₃ and 2%_{(aq)} Solution

The oxidized form of hyaluronan (100.0 mg, 0.265 mmol, DS=20%, Mw=34.4 kDa) is dissolved in 5 ml of distilled water (2% solution). To said solution, AEP (11.0 mg, 0.080 mmol, 1.5 eq) and NaHCO₃ (22.2 mg, 0.265 mmol) are added. The reaction mixture is stirred for 2 hours. Then NaBH₃CN (26.5 mg, 0.425 mmol) is added and the reaction mixture is stirred for further 12 hours. The final solution is dialysed and lyophilized.
DS=17%, Mw=31 kDa, isolated yield 79% (determined by NMR, more details in Example 6)

### Example 9

### Or Photocrosslinking of the Photoreactive Derivatives of Hyaluronic Acid—Method A

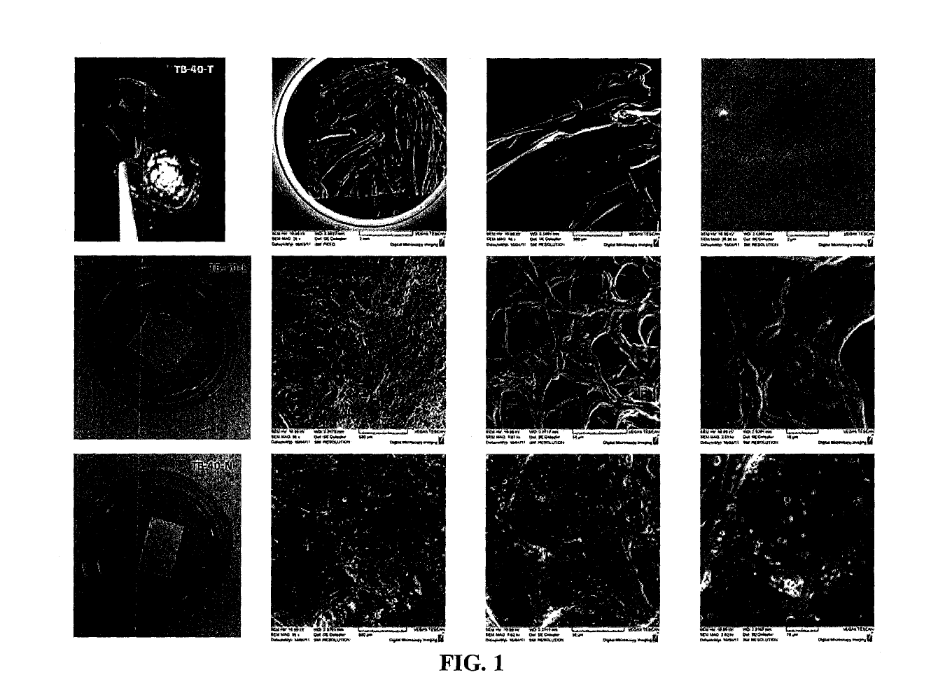

The irradiated material as thin film was prepared by the evaporation of a 5%_{(aq)} solution of the photoreactive derivative of hyaluronic acid (DS=18%, Mw=25 kDa). The solution was transferred into Petri dishes and evaporated in a hot-air drier at 40° C. for 12 hours. The prepared thin film was placed on an aluminium foil in a Petri dish and irradiated for 1 hour (E=24300 mJ/cm²). After the exposition of the material, the change of the physical properties thereof was tested (solubility and stability) compared to the non-irradiated sample. The analysis was carried out in distilled water and PBS (pH=7) at 25° C. The undissolved material was filtrated off and lyophilized for SEM analysis. The filtrate was evaporated and analysed by NMR. Results of the tests and of the NMR analysis of extracts of the exposed material are presented in Table 1. The photographic analysis is presented in FIG. 1.

TABLE 1

| | | | | solubility/stability | | | | | NMR |
|---|---|---|---|---|---|---|---|---|---|
| | DS | Mw | E | time [h] | | | | | extract |
| Sample | [%] | [kDa] | [mJ · cm⁻²] | 12 | 24 | 36 | 48 | gelation/swelling | HA/PEO |
| TB-40-L | 18 | 25 | 24300 | −/+ | −/+ | −/+ | −/+ | +/+ | −/− |
| TB-40-N | 18 | 25 | 24300 | −/+ | −/+ | −/+ | −/− | +/+ | −/+ |
| TB-40-T | 18 | 25 | 24300 | −/+ | −/+ | −/+ | −/+ | +/+ | −/− |
| TB-39-L | 20 | 15 | 24300 | −/+ | −/+ | −/+ | −/+ | +/+ | −/− |
| TB-31-N | 20 | 34 | 24300 | −/+ | −/+ | −/− | −/− | +/+ | −/+ |
| TB-23-L | 3 | 229 | 24300 | −/+ | −/+ | −/+ | −/+ | +/+ | −/− |

Results of an analysis of photocrosslinked derivatives of hyaluronic acid in H₂O and PBS (pH = 7.4).
L—lyophfilized form,
N—nanofibrous layer,
T—thin film.
+ represents a positive result and
− represents a negative result.

### Example 10

### Photocrosslink of the Photoreactive Derivatives of Hyaluronic Acid—Method B

The irradiated material was in the form of a lyophilizate which was prepared by lyophilization of a 5%_{(aq)} solution of the photoreactive derivative of hyaluronic acid (DS=18%, Mw=25 kDa). A thin layer (approx. 0.5-1.0 mm thick) and dimensions (2×2 cm) of the lyophilizate was placed on an Al foil in a Petri dish. The lyophilizate was irradiated for 1 hour (E=24300 mJ/cm²). After the exposition of the material, the change of the physical properties thereof (solubility and stability) was tested compared to the non-irradiated sample. The analysis was carried out in distilled water and phosphate buffer (PBS—phosphate buffered saline, pH=7) at 25° C. The undissolved material was filtrated off and lyophilized for SEM (scanning electron microscope) analysis. The filtrate was evaporated and analysed by NMR. Results of the tests and of the NMR analysis of extracts of the exposed material are presented in Table 1.The photographic analysis is presented in FIG. 1.

### Example 11

### Photochemical Crosslinking of the Photoreactive Derivatives of Hyaluronic Acid—Method C

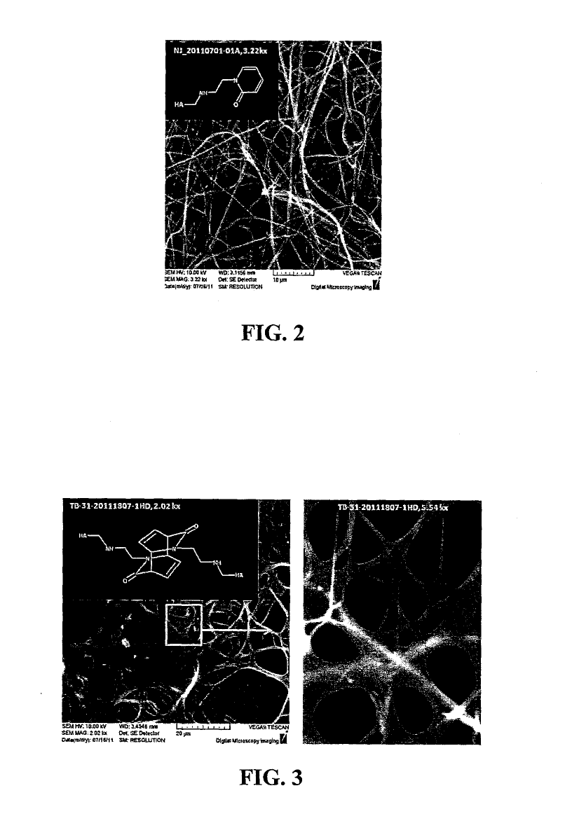

The irradiated material is in the form of a nanofibrous layer having an average basis weight of 0.3 mg/cm². The nanofibrous layer was prepared by electrostatic spinning (electrospinning) by use of apparatus 4Spin made by Contipro Biotech s.r.o. The concentration of the spinned aqueous solution was 10% by weight. The realtive weight ratio of the photoreactive polymer of hyaluronic acid (DS=18%, Mw=25 kDa) and the supportive polymer polyethyleneoxide (Mw=600 kDa) was (80/20). The nanolayers coated on the polypropylene basement textile with the size of (2×2 cm) were placed on an aluminium foil in a Petri dish. The material was irradiated for 1 hour (E=24300 mJ/cm²). After the exposition of the material, the change of the physical properties thereof (solubility and stability) was tested compared to the non-irradiated sample. The analysis was carried out in distilled water and PBS (pH=7) at 25° C. The undissolved material was filtrated off and lyophilized for SEM analysis. The filtrate was evaporated and analysed by NMR. Results of the tests and of the NMR analysis of extracts of the exposed material are presented in Table 1. The photographic analysis is attached (FIGS. 1, 2, 3).

### Example 12

### Tests of Cell Viability of the Photoreactive Derivatives

The tested substance (DS=18%, Mw=25 kDa) was dissolved in complete 3T3 cell culture medium. The solution was filtrated through a filtration device (0.22 μm). The final testing concentrations of the solution were 100, 500, 1000 μg/ml. 3T3 cells having the density of 3 000 cells per a well were seeded to wells of 96-well test plates. Prior to test, the cells were cultivated for 24 hours in the complete cell medium. The cell viability was measured by means of the 3-[4,5-dimethylthiazol-2-yl]-2,5-diphenyl tetrazolium bromide (MTT) method in intervals 0, 24, 48, 72 hours. In the assay, MTT is reduced by viable cells to a purple coloured water-insoluble formazane, which is later determined by the spectrophotometry.

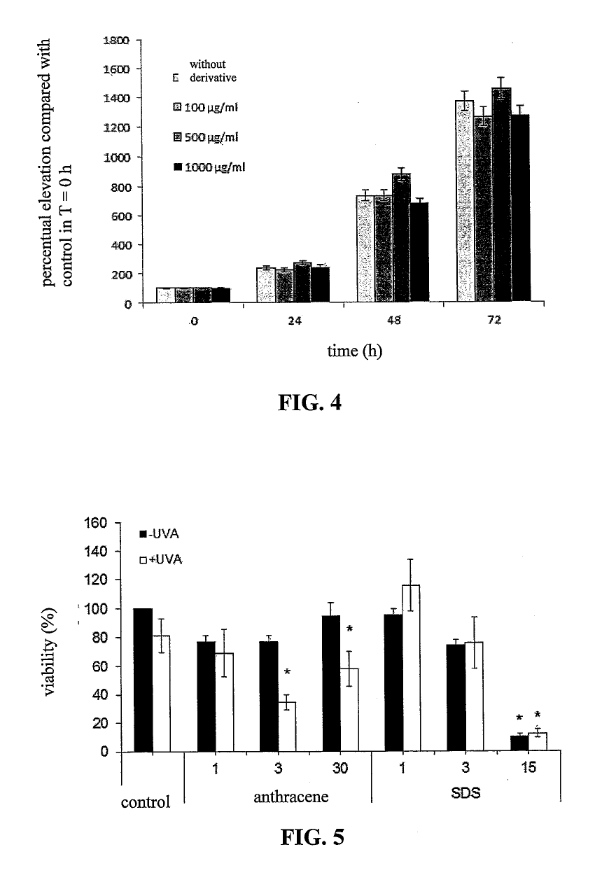

20 μl of MTT stock solution (5 mg/ml) were added to 200 μl of the cell culture medium in each well. The plates were incubated at 37° C. in a termoregulator for 2.5 hours. Then the solution above the cells was sucked off and the solubilizing solution having the volume of 220 μl was added. The optical density of the solution was measured by Microplate reader VERSAmax at 570 nm (690 nm background). The whole experiment was supplemented by a number of non-influenceable controls and blank samples. Based on the measured data of optical density, percentual representation relating to the control in time T0 hours was calculated (ratio of the optical density of the influenceable sample to the optical density of the non-influenceable control T0, multiplied by 100) and the standard deviation of the average (SEM). The results of the viability test are graphically processed in the attachment (FIG. 4).

### Example 13

### Tests of the Phototoxicity of the Photoreactive Derivatives

3T3 cells having the density of 10 000 cells per a well were seeded into 96-wells panels. Prior to test, the cells were cultivated for 24 hours in the complete cell medium. Then the cells were washed with PBS (pH=7.00) and incubated for 1 hour with the tested substances dissolved in PBS (tested substance TB-13: DS=20%, Mw=34 kDa, 1, 3, 30, 100, 500, 1000, 5000 μg/ml; phototoxic anthracene-1, 3, 30 μg/ml; non-phototoxic SDS-1, 3, 15 μg/ml). The cells were irradiated with the dose of 0.1 J/cm² UVA (315-400 nm) using a lamp (Oriel Instruments) and the output thereof was determined by a photometer PMA 2100 (Solar light Co.). 10 minutes after the exposition, the supernatant was removed from the cells and the complete cell medium was added. The cell viability was evaluated spectrophotometrically by means of the MTT method 24 hours after the irradiation. The results of the test are graphically processed in the attachment (FIGS. 5 and 6).

### Example 14

### Tests of Biodegradability of the Photocrosslinked Derivatives

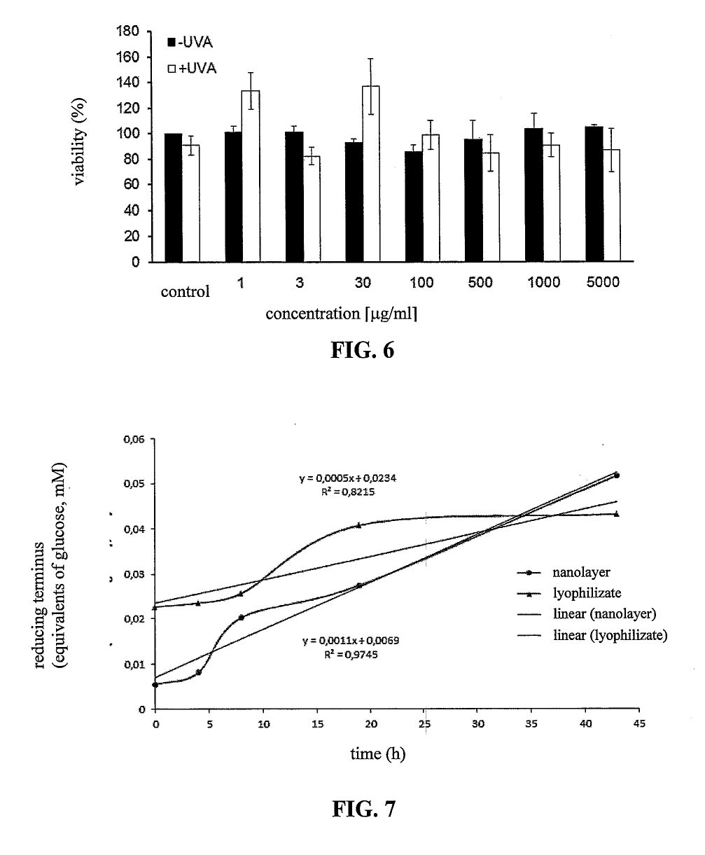

The photocrosslinked derivatives of hyaluronic acid: the lyophilizate (L)=16.3 mg and the nanolayer form: m (N)=9.0 mg were prepared in sterile conditions, overlaid with 2 ml of PBS (pH=7.38) and swelled for 24 hours. To each sample, 200 U of BTH (BTH=bovine testicular hyaluronidase, EC 3.2.1.35) were added and the samples were incubated for 43 hours at 37° C. In time intervals 0, 4, 8, 19 and 43 hours, 100 μl of each sample were taken away and maintained at −20° C. until the final analysis. At the same time, the controls (PBS+BTH and the pure derivatives in PBS) were incubated. The absorbance of the control PBS+BTH was subtracted as background 1. The absorbance of the buffer with the derivative after swelling (time T=0) was subtracted as background 2. At the same time the control with pure derivative without any enzyme in the pure PBS was incubated in order to find out whether the sample undergoes a spontaneous degradation. The free reducing ends were determined by means of Somogyi and Nelson test by the following procedure: 50 μl of the sample were mixed with the same volume of freshly prepared Somogyi reagent. After mixing, the mixture was incubated in a thermoblock for 15 minutes at 100° C. After cooling, 100 μl of Nelson agent were added, the samples were mixed, centrifuged and their absorbance at 540 nm was determined. After subtracting the background, the values of glucose equivalents (analogy of free reducing ends) were determined from the calibration curve. The results of the test are graphically processed in the attachment (FIG. 7).

1. A photoreactive derivative of hyaluronic acid according to the formula (I), wherein R represents hydrogen or an alkali metal cation

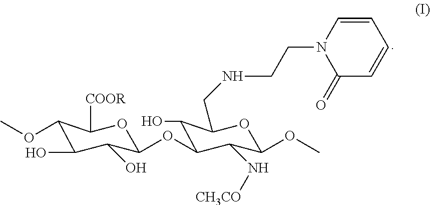

2. The photoreactive derivative of hyaluronic acid according to claim 1, where hyaluronic acid or an inorganic salt thereof has the molecular weight within the range of 1.10⁴ to 5.10⁶ g.mol⁻¹.

3. A method of preparation of the photoreactive derivative defined in any of the preceding claims, characterized by that first an aldehydic derivative of hyaluronic acid oxidized in the position 6 of the glucosamine cycle is prepared and then the oxidized derivative is reacted with an amine carrying a photoreactive species in the presence of a reducing agent, forming the photoreactive derivative.

4. The method of preparation according to claim 3, characterized by that for the selective preparation of the aldehyde in the position 6 of the glucosamine part of hyaluronic acid, the oxidation agent Dess-Martin periodinane in an aprotic medium is used or TEMPO radical with NaClO in an aqueous medium is used.

5. The method of preparation of the photoreactive derivative according to claim 3 or 4, characterized by that the aldehydic derivative of hyaluronic acid reacts with an amino group of the amine carrying the photoreactive species, forming an imine which is directly reduced in one step in the presence of the reducing agent NaBH₃CN in an aqueous medium or in water-organic solvent system to a secondary amine.

6. The method of preparation of the photoreactive derivative according to any of claims 3 to 5, characterized by that the amine carrying the photoreactive species is 1-(2-aminoethyl)pyridine-2(1H)-one.

7. The method of preparation of the photoreactive derivative according to claims 3 to 6, characterized by that DS of the oxidized hyaluronic acid is within the range from 1 to 40%, and DS of the secondary amine carrying the photoreactive species according to claim 6 is within the range from 1 to 40%, preferably 15 to 20%.

8. The method of preparation of the photoreactive derivative according to any of claims 3 to 7, characterized by that 1-2% wt. aqueous solution of the aldehydic derivative of hayluronic acid is prepared, 1-2 eqs of the amine carrying the photoreactive species are added and then 1-3.5 eqs of the reducing agent NaBH₃CN are added, forming the photoreactive derivative according to the formula (I).

9. The method of preparation of 3D-crosslinked derivatives of hyaluronic acid, characterized by that the photoreactive derivative defined in any of claims 1 to 2 is exposed to electromagnetic radiation within the wavelengths of 280-315 nm.

10. The method of preparation according to claim 9, characterized by that the photoreactive derivative is in the form of a powder, lyophilizate, thin film, a nanofibrous or microfibrous structure.

11. A 3D-crosslinked derivative of hyaluronic acid according to the formula (II):

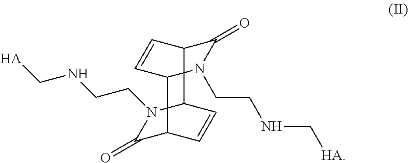

12. Use of the 3D-crosslinked derivative defined in claim 11 for tissue engineering, regenerative medicine, medical devices or cosmetics.

13. Use of the 3D-crosslinked derivative defined in claim 11 as scaffolds, fillings, drug carriers, support nano- or micro-structures for cell growth, especially of stem cells or differentiated cells of the type of chondrocytes, fibroblasts, neurocytes and the like, for the preparation of nano- or micro-structures, woven fabrics, knitted fabrics for the production of biodegradable bandages with controlled release of biologically active substances for surface wounds, for the production of facial masks or as an additive to sun lotions with a preventive or regenerative effect.
